(19) United States
(12) Patent Application Publication
(10) Pub. No.: US 2022/0142272 A1
(43) Pub. Date: May 12, 2022

(54) NASAL MASK METHOD AND DEVICES

(71) Applicant: FRANK BARONE, Chula Vista, CA (US)

(72) Inventor: FRANK BARONE, Chula Vista, CA (US)

(21) Appl. No.: 17/094,905

(22) Filed: Nov. 11, 2020

(51) Int. Cl. A41D 13/11; G01N 33/00

(52) U.S. Cl. CPC A41D 13/1169; A41D 13/1107; A41D 2500/30; G01N 33/0075; A41D 13/1192

(57) ABSTRACT

The embodiments disclose a method including fabricating a nasal mask configured for preventing nasal inhaling of airborne Covid-19 virus, a self-adhesive nasal strip mask for inactivating air borne viruses being inhaled by a user, making the nasal mask using a breathable fabric configured for creating a breathable fabric filter for filtering out air borne viruses, providing at least two ear straps coupled to the nasal mask configured for a user looping the at least two ear straps over the user's ears, integrating a dorsum conforming nose piece coupled to the nasal mask configured for creating a gap free fit, and fabricating structural stitching in the nasal mask configured for maintaining a breathable space fronting the nostrils.

### BACKGROUND

Recent medical and scientific studies have found that air borne viruses are more likely to thrive in the nasal passages than the mouth and oral areas. Current masks concentrate on protecting the mouth. The half-face mask coverings can cause users difficulty in breathing for extended period and make conversations difficult to hear and understand.

### BRIEF DESCRIPTION OF THE DRAWINGS

FIG. 1 shows for illustrative purposes only an example of an overview of a nasal mask worn by a user of one embodiment.

FIG. 2A shows for illustrative purposes only an example of nostril inserts in nasal passages of one embodiment.

FIG. 2B shows for illustrative purposes only an example of a self-adhesive nasal strip of one embodiment.

FIG. 3A shows for illustrative purposes only an example of mask space extending structure of one embodiment.

FIG. 3B shows for illustrative purposes only an example of open weave anti-microbial barrier of one embodiment.

FIG. 4A shows for illustrative purposes only an example of Bluetooth device of one embodiment.

FIG. 4B shows for illustrative purposes only an example of virus detection sensor with near-field transmitter of one embodiment.

FIG. 5 shows for illustrative purposes only an example of extended breathing space of one embodiment.

FIG. 6 shows for illustrative purposes only an example of dorsum conforming nose piece of one embodiment.

FIG. 7 shows for illustrative purposes only an example of nasal mask filtered air inhaled of one embodiment.

FIG. 8 shows for illustrative purposes only an example of nostril insert anti-microbial barrier destroying air borne virus of one embodiment.

FIG. 9 shows a block diagram of an overview of an articulated mouth mask of one embodiment.

FIG. 10A shows for illustrative purposes only an example of an articulated mouth mask down of one embodiment.

FIG. 10B shows for illustrative purposes only an example of an articulated mouth mask raised of one embodiment.

FIG. 11 shows a block diagram of an overview of a nasal spray application of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview:

It should be noted that the descriptions that follow, for example, in terms of a nasal mask method and devices is described for illustrative purposes and the underlying system can apply to any number and multiple types of nasal masks. In one embodiment of the present invention, the nasal mask method and devices can be configured using electronic devices. The nasal mask method and devices can be configured to include supporting structures and can be configured to include nasal insert using the present invention.

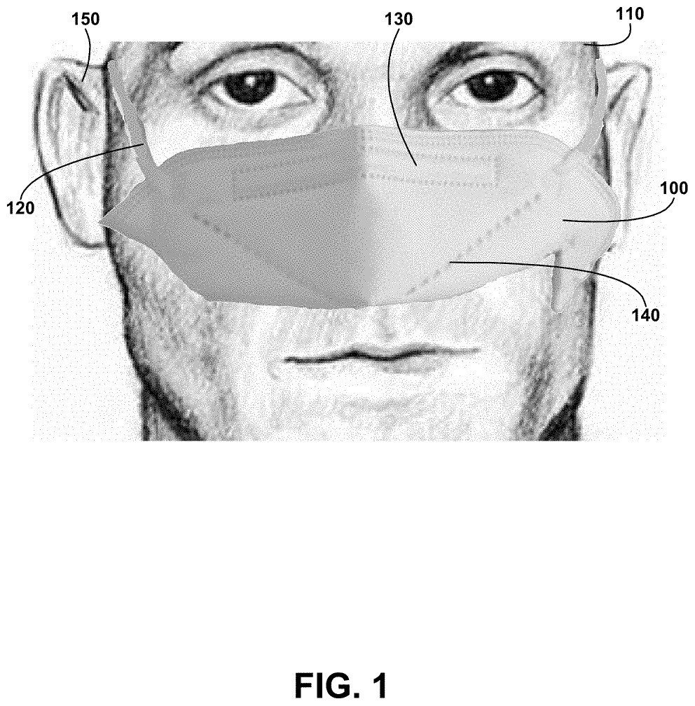

FIG. 1 shows for illustrative purposes only an example of an overview of a nasal mask worn by a user of one embodiment. FIG. 1 shows a nasal mask 100 being worn by a user 110. The nasal mask 100 includes an ear strap 120 on the right and left sides for looping behind the right and left outer ear 150. A dorsum conforming nose piece 130 is pressed by the user 110 to form a tight fitting seal across and down both sides of the dorsum. Structural stitching 140 are positioned to support the nasal mask 100 sections formed around the nasal nostril features. In one embodiment the nasal mask 100 comprises a breathable fabric allowing the user to comfortable inhale and exhale through the nasal mask 100. The nasal mask breathable fabric forms a breathable fabric for preventing air borne virus from being inhaled through the nostrils. In one embodiment the breathable fabric is coated with an antiseptic providone-iodine solution with a concentration ranging from 0.5% to 2.5% for inactivating the SARS-CoV-2 virus. In another embodiment the breathable fabric is coated with disinfectant for example a bromine chloride solution with a concentration of 28 μm to 170 μm for inactivating the SARS-CoV-2 virus of one embodiment.

### DETAILED DESCRIPTION

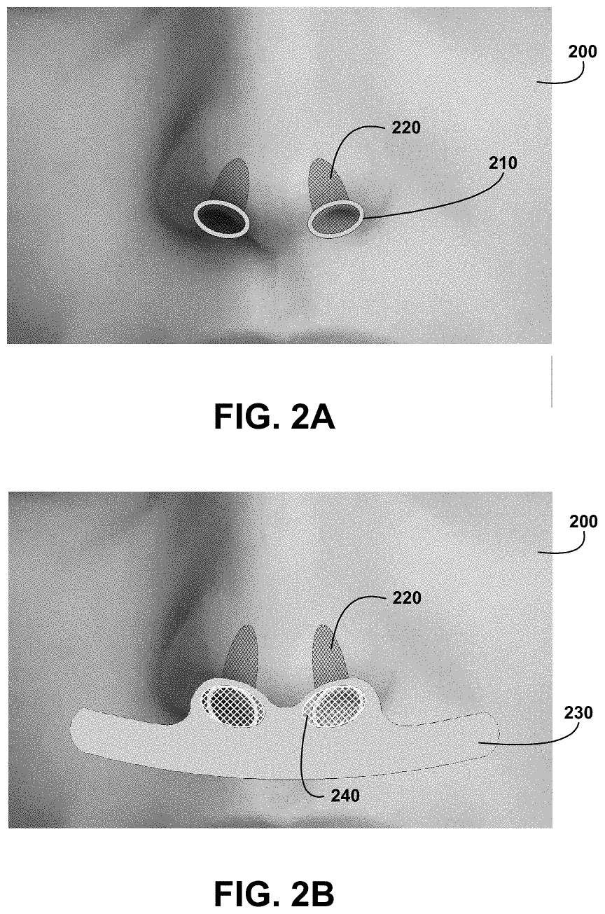

FIG. 2A shows for illustrative purposes only an example of nostril inserts in nasal passages of one embodiment. FIG. 2A shows a user's nose and cheek area 200 of the face. A nostril insert 220 shown fully visible for illustrative purposes only inserted into the nostril. A nostril insert ring 210 has an adhesive applied to adhere the nostril insert 220 to the exterior of the nostril. The nostril insert 220 is a disposable application based device. A user inserts the nostril insert 220 for a specified period of time based on the antimicrobial material infused and coated into the nostril insert 220 breathable material used to fabricate the insertion portion of the nostril insert 220. The nostril insert 220 is packaged in a hermetically sealed package. When the user unseals the package the antimicrobial material(s)s are activated and absorbed into the breathable material. The user inserts the nostril insert 220 with the now antimicrobial material infused and coated breathable material. The nostril insert 220 is infused and coated with an antimicrobial for example bromine, iodine and hypochlorous acid. The absorbent material of the nostril insert 220 holds the antimicrobial material in a solution. In one embodiment the antimicrobial material is a gel. In another embodiment the user upon inserting the nostril inserts 220 in both nostrils squeezes (pinches) the outside of the nostrils to apply the absorbed antimicrobial material to the inside surfaces of the nostrils. After applying the antimicrobial material coating to the inside surfaces of the nostrils the user may remove the disposable nostril inserts 220 of one embodiment. The user may insert nostril inserts 220 and cover the nostril inserts with a self-adhesive nasal strip mask 230 of FIG. 2B.

### Self-Adhesive Nasal Strip Mask:

FIG. 2B shows for illustrative purposes only an example of a self-adhesive nasal strip mask of one embodiment. FIG. 2B shows a user's nose and cheek area 200 of the face. A nostril insert 220 shown fully visible for illustrative purposes only inserted into the nostril. The nostril insert 220 in each nostril is covered with the self-adhesive nasal strip mask 230. The self-adhesive nasal strip mask 230 further comprises a nasal filter 240. The inside surfaces against the skin include an adhesive to hold the self-adhesive nasal strip mask 230 in a position placing the nasal filter 240 at each nostril opening. The nasal filter 240 is a multi-layer filtration system that filters out at least 95% of airborne particles. The filters trap large and small particles including viruses, bacteria, microorganisms and fungi. The multi-layer filters are also coated with antimicrobial materials, biocides and other treatments to reduce and/or kill microorganisms, or viruses including the SARS-CoV-2 virus, or bacteria, or fungi, or affecting allergenicity. In one embodiment the nasal filter 240 breathable material is infused and coated with an antimicrobial for example bromine, iodine and hypochlorous acid.

The filters are made from breathable material allowing the user to easily inhale and exhale. In another embodiment the multi-layer filtration system comprises non-woven breathable fibers including non-woven polypropelene, melt-blown fabric, and ES hot air cotton.

The nasal filter 240 breathable material is absorbent and holds the antimicrobial material in a solution. In one embodiment the antimicrobial material is a gel. When a user inhales the nasal filter 240 breathable material infused and coated with the antimicrobial material inactivates air borne viruses and prevents the viruses from replicating in the nasal cavity. The self-adhesive nasal strip mask 230 is disposable. The self-adhesive nasal strip mask 230 is wearable underneath a facial mask of one embodiment.

Mask Space Extending Structure:

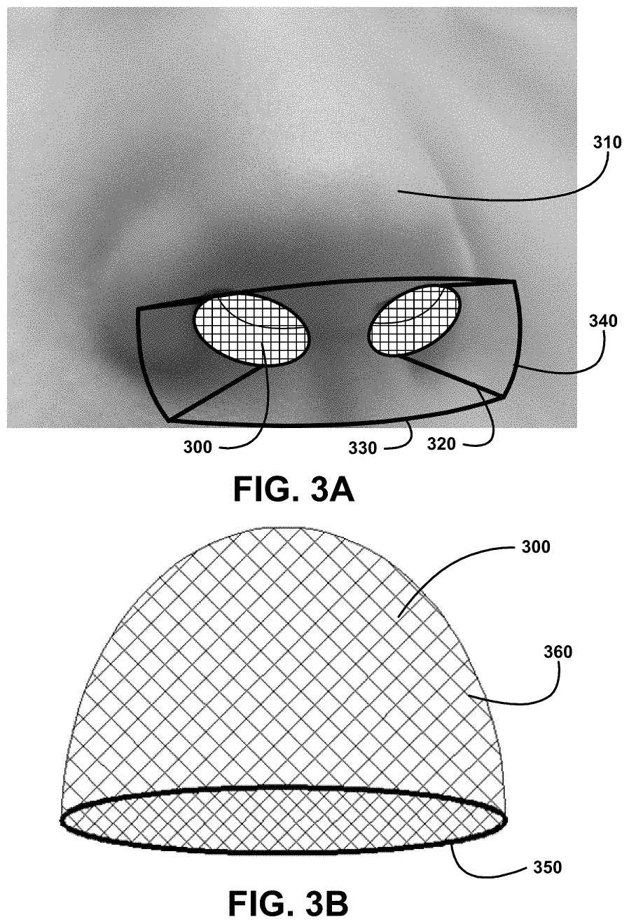

FIG. 3A shows for illustrative purposes only an example of mask space extending structure of one embodiment. FIG. 3A shows a user nose 310 and in one embodiment a nostril insert 300 positioned in both nostrils. Each nostril insert 300 is coupled to two nostril insert connector 320 structures. The two nostril insert connector 320 structures are coupled to two or more horizontal brace 330 structures and two or more vertical brace 340 structures. The nostril insert connector 320 structures, horizontal brace 330 structures and vertical brace 340 structures form a mask space extending structure. The mask space extending structure elements are made of a semi-rigid plastic. In one instance the mask space extending structure extends the breathable fabric filter 220 away from the nasal opening to provide additional breathing space for the user. In another instance the extended breathable fabric filter 220 applies gentle pressure against the mask space extending structure to maintain the inserted positons of each nostril insert 300 of one embodiment.

Open Weave Anti-Microbial Barrier:

FIG. 3B shows for illustrative purposes only an example of open weave anti-microbial barrier of one embodiment. FIG. 3B shows the nostril insert 300. The nostril insert 300 includes an elliptical flexible nostril stop 350 configured with a thin tapered flexible skirting for sealing against the outside of the nostril. The half-spheroidal insert is an open weave semi-rigid material with an anti-microbial coating. The anti-microbial coating includes for example bromine, iodine and other non-toxic, non-allergenic biocides. The nasal mask in one embodiment includes a coating of an antiseptic providone-iodine solution with a concentration ranging from 0.5% to 2.5%. In another embodiment coating the breathable fabric filter with a disinfectant for example a bromine chloride solution with a concentration of 28 μm to 170 μm for inactivating the SARS-CoV-2 virus.

The nostril insert 300 and anti-microbial coating form an open weave anti-microbial barrier 360 for destroying air borne viruses before entering the nasal cavity. Medical studies show at least 90% of inhaled air enters the body through the nose. Medical and scientific studies show the nasal cavity is likely the major site of entry and infection by Covid-19. Nasal virus production is at very high levels and tends to occur early in the disease process while patients are still asymptomatic or having very mild symptoms.

In one embodiment at least one ultraviolet LED with a power source is coupled to the nostril insert for disinfecting the nasal cavity. The at least one ultraviolet LED is used to radiate ultraviolet light into the nostrils and nasal cavity. The at least one ultraviolet LED uses a light wave length and power level that will not damage the sensitive tissues of the nostrils and nasal cavity. The at least one ultraviolet LED in one embodiment has a timer coupled to the at least one ultraviolet LED that will turn on the ultraviolet radiate for a predetermined period of time and then automatically turn off. The on/off cycle is repeated automatically at predetermined intervals to maintain a disinfected status of the nostrils and nasal cavity. The ultralight radiation disrupts virus, bacteria and microorganism DNA and disables an ability to replicate. This will assist in preventing infection by viruses including the SARS-CoV-2 virus and influenza virus.

In another embodiment the user can use soap and water for cleaning a nostril insert fabricated from a sponge material. In yet another embodiment nostril inserts are disposable. The nostril inserts are made of a re-useable material that the user can disinfect and clean of one embodiment.

In another embodiment the nostril insert is dipped into or moistened with an antimicrobial solution and used to swab the nostril opening with the antimicrobial solution for inactivating the SARS-CoV-2 virus.

### Bluetooth Device:

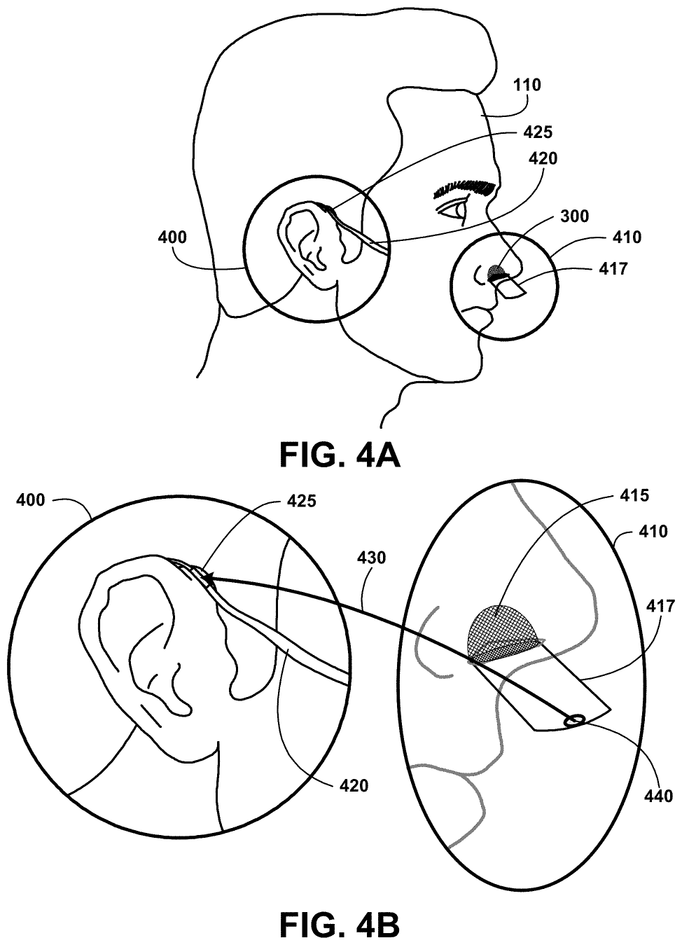

FIG. 4A shows for illustrative purposes only an example of Bluetooth device of one embodiment. FIG. 4A shows the user 110 with only two sections of one embodiment of the nasal mask 100 of FIG. 1 shown. In Detail “A” 400 is shown an ear strap 420 where it loops around the top of the outer ear. The embodiments of the nasal mask, nostril inserts and self-adhesive nasal strip devices include digital modules to allow the user to receive audible alerts and alarms of potential infectious conditions.

The digital modules include sensors, detectors and meters are configured for detecting and measuring the conditions surrounding the user and other factors that affect the user's exposure to bacteria, viruses and fungi. The digital modules including the sensors, detectors, meters and communication devices are coupled to the embodiments of the nasal mask, nostril inserts and self-adhesive nasal strip devices. The digital modules are wirelessly coupled to a nasal mask app for transmitting detected and measured data to the nasal mask app for analysis and processing to determine an exposure risk level in the conditions surrounding the user to trigger the wireless transmission of an alert alarm to the user. The communication devices include WIFI and cellular connectivity.

The nasal mask app is installed on a user's digital device for example a smart phone. The nasal mask app includes stored identification data on numerous bacteria, viruses and fungi that is automatically updated daily. The digital modules sensors and detectors include biological and chemical sensing and detecting capabilities. The data collected by the sensors and detectors is wirelessly transmitted to the nasal mask app. The nasal mask app processes the biological and chemical data for analysis. The analysis determines a match to a stored identified bacteria, viruses and fungi.

The additional conditions surrounding the user are analyzed to determine for example proximity to other persons, wind and air flow conditions that affect spreading of air-borne pathogens, temperature that affect viability of air-borne pathogens, humidity that can affect viability of air-borne pathogens for generating a risk assessment of potential infectious conditions. The risk assessment analytical results are transmitted to a user's digital device for example a smart phone audible and display and Bluetooth communication device. Included in the alert are suggested actions a user can take to move locations based on their GPS location, wind direction and proximity to other persons to reduce any determined risks.

Coupled to the ear strap 420 is a Bluetooth device 425. Showing in Detail “B” 410 is the nostril inserts 300 and mask space extending structure 417 of one embodiment.

Virus Detection Sensor with Near-Field Transmitter:

FIG. 4B shows for illustrative purposes only an example of virus detection sensor with near-field transmitter of one embodiment. FIG. 4B shows Detail “A” 400 with the ear strap 420 and Bluetooth device 425. Detail “B” 410 shows the nostril insert 300 and mask space extending structure 417. A virus detection sensor with near-field transmitter 440 is coupled to the mask space extending structure 417. In another embodiment the virus detection sensor with near-field transmitter 440 is integrated into the nasal mask 100 of FIG. 1 in front of the nostrils. The virus detection sensor is shown transmitting signal to the Bluetooth device 425. This transmission received by the Bluetooth device 425 activates an audible alert that a coronavirus has been detected. The low volume audible alert can be heard by the user 110 due to the proximity of the Bluetooth device 425 to the user's ear without causing a disturbance to others around the user 110. Upon hearing the audible alert the user 110 may decide to move locations to reduce exposure to the virus in the air of one embodiment.

### Extended Breathing Space:

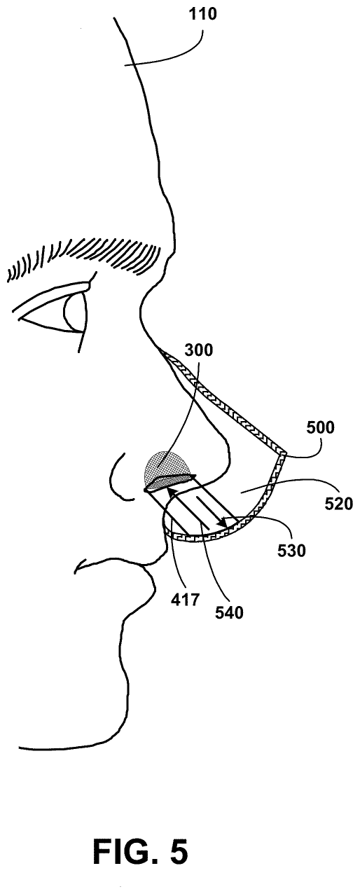

FIG. 5 shows for illustrative purposes only an example of extended breathing space of one embodiment. FIG. 5 shows the user 110 in a profile with a nostril insert 300 and mask space extending structure 417. The nasal mask 100 of FIG. 1 is only partially shown in a breathable fabric filter cross section 500. The mask space extending structure 417 extending outward from the nostrils and nostril insert 300 create an extended breathing space 520 in front of the nostrils. The extended breathing space 520 allows the user 110 to breathe more comfortably through the nose. Nasal inhaling 540 may draw the nasal mask 100 of FIG. 1 inward. The structural stitching 140 will reduce the opportunity of the nasal mask 100 of FIG. 1 from being drawn in when the user 110 is nasal inhaling 540. When the user 110 is nasal exhaling 530 the exhaled air will pass through the breathable fabric filter 220 of FIG. 2 easily of one embodiment.

### Dorsum Conforming Nose Piece:

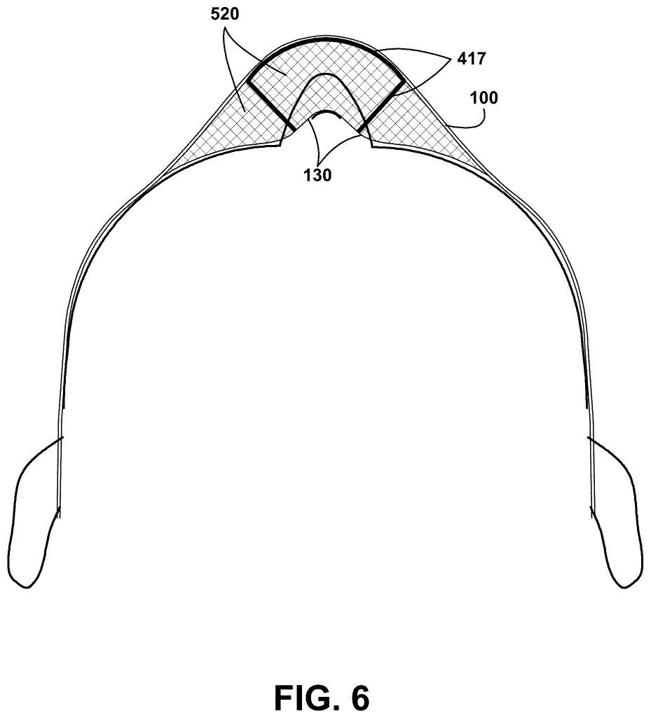

FIG. 6 shows for illustrative purposes only an example of dorsum conforming nose piece of one embodiment. FIG. 6 shows the dorsum conforming nose piece 130 where the user 110 of FIG. 1 has pressed the dorsum conforming nose piece 130 against the nose structure and created a tight fitting contact to no gaps that would allow unfiltered air from entering. The mask space extending structure 417 is shown holding the nasal mask 100 away from the nostril area and creating the extended breathing space 520 of one embodiment.

### Nasal Mask Filtered Air Inhaled:

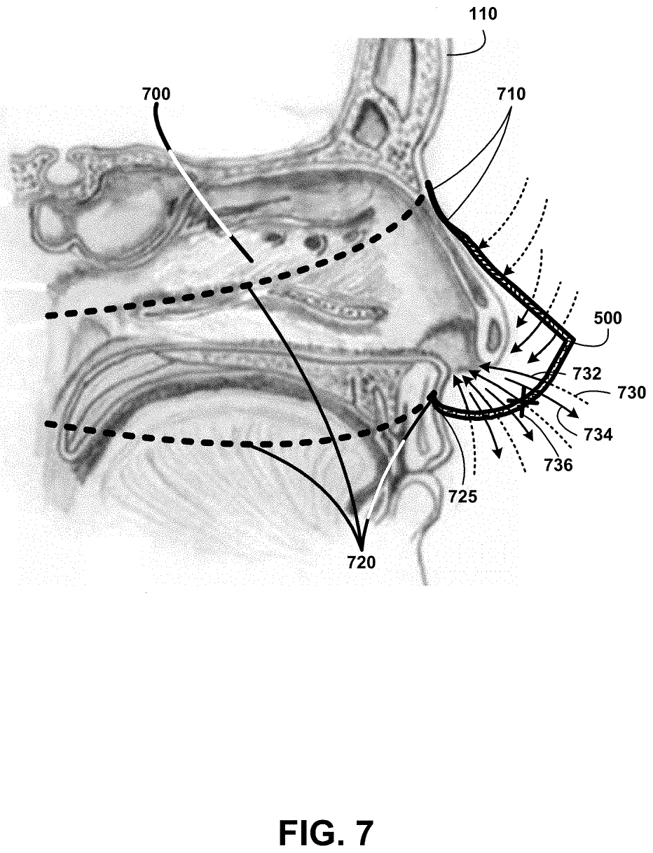

FIG. 7 shows for illustrative purposes only an example of nasal mask filtered air inhaled of one embodiment. FIG. 7 shows the user 110 in a nasal structure diagram cut away illustrating the nasal cavity 700 and nasal passages. The dorsum conforming nose piece no gap fit 710 illustrates the contact fit of this section of the nasal mask 100 of FIG. 1. Also illustrated is the nasal mask face no gap fit 720 along the sides of the user face and nasal mask upper lip no gap fit 725. The public is wearing in general loose fitting face masks that allow air to pass around the mask from all sides and get inhaled through the mouth and nose. Covid-19 is spread through an air borne virus and the majority of commercially inexpensive face masks do little to prevent a user from coming in contact with the air borne virus. Shown is a breathable fabric filter cross section 500. Illustrated as dotted lines is ambient air inhaled with possible airborne virus 730. The firm fitting no gap nasal mask 100 of FIG. 1 nasal mask filtered air inhaled 732 prevents air borne virus from being inhaled. Additionally for a user 110 of FIG. 1 who may be infected but asymptomatic the breathable fabric filter exhaled air 734 will prevent the user from spreading the infection to others. The nasal mask 100 of FIG. 1 breathable fabric filter preventing entry to air borne virus 736 will reduce the spread of the Covid-19 of one embodiment.

### Nostril Insert Anti-Microbial Barrier Destroying Air Borne Virus:

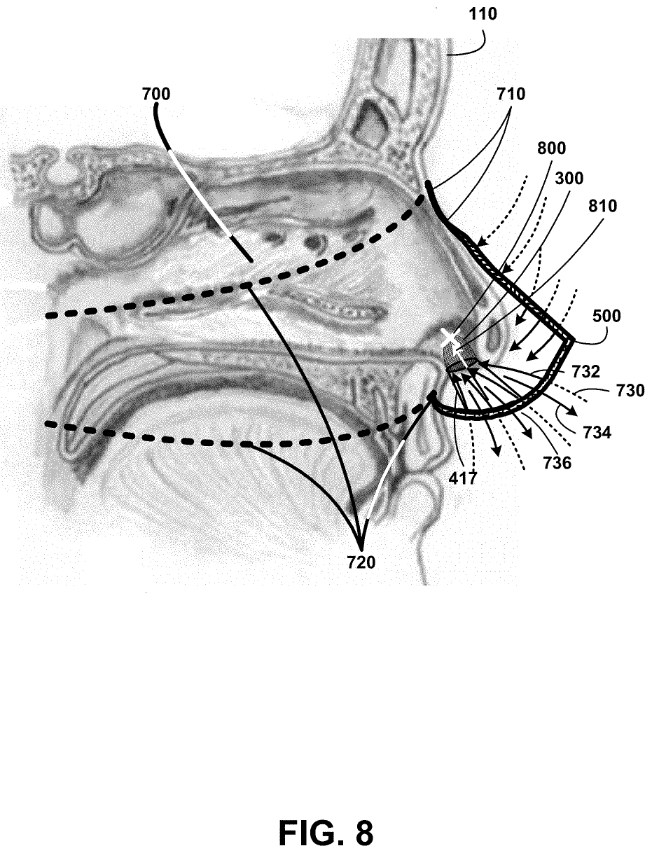

FIG. 8 shows for illustrative purposes only an example of nostril insert anti-microbial barrier destroying air borne virus of one embodiment. FIG. 8 shows another embodiment of the nasal mask 100 of FIG. 1. The user 110 in a nasal structure diagram cut away illustrating the nasal cavity 700 and nasal passages. The dorsum conforming nose piece no gap fit 710 illustrates the contact fit of this section of the nasal mask 100 of FIG. 1. Also illustrated is the nasal mask face no gap fit 720 along the sides of the user face and nasal mask upper lip no gap fit 725.

There are numbers within the population who have preexisting conditions that will lead to severe complications if the members of the population get infected with Covid-19. These higher at risk segments of the population may be helped in their efforts by wearing the nasal mask 100 of FIG. 1. In this example the breathable fabric filter cross section 500 will stop ambient air inhaled with possible airborne virus 730 with the nasal mask filtered air inhaled 732.

The higher at risk individuals may be further assisted in their efforts to prevent infection with the nostril insert 300. The inhaled air passing through nostril insert 810 will provide a second layer of protection with the nostril insert anti-microbial barrier destroying air borne virus 800. This addition coupled with the breathable fabric filter exhaled air 734 for example will help in the two-way reduction of spreading the infection. The breathable fabric filter preventing entry to air borne virus 736 coupled with the mask space extending structure 417 assisting with extended breathing space 520 of FIG. 5 will allow those at higher risk to breathe more comfortably and have less reason to not wear any mask. The discussions of the medical and scientific studies show at least 90% of inhaled air enters the body through the nose. The firm fitting nasal mask 100 of FIG. 1 will be a vast improvement by reducing the chances of inhaling an air borne virus to 10% or less.

### An Articulated Mouth Mask:

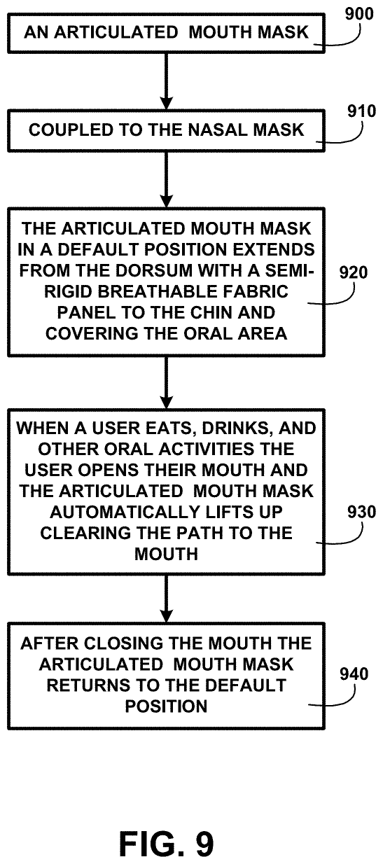

FIG. 9 shows a block diagram of an overview of an articulated mouth mask of one embodiment. FIG. 9 shows an articulated mouth mask 900 coupled to the nasal mask 910. The articulated mouth mask in a default position extends from the dorsum with a semi-rigid breathable fabric panel to the chin and covering the oral area 920. When a user eats, drinks, and other oral activities the user opens their mouth and the articulated mouth mask automatically lifts up clearing the path to the mouth 930. After closing the mouth the articulated mouth mask returns to the default position 940 of one embodiment.

### An Articulated Mouth Mask Down:

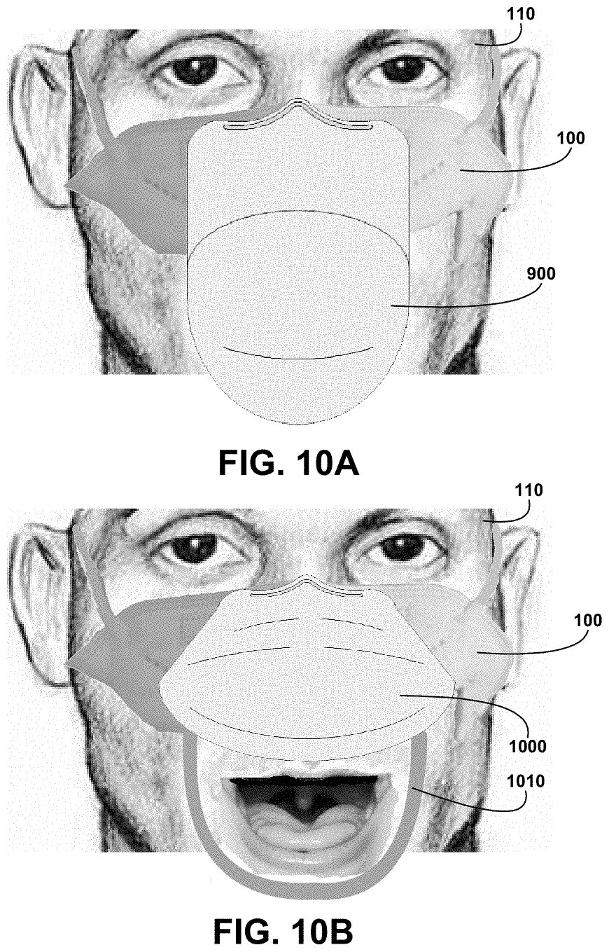

FIG. 10A shows for illustrative purposes only an example of an articulated mouth mask down of one embodiment. FIG. 10A shows the user 110 wearing the nasal mask 100 configured with a coupled articulated mouth mask 900 in the default position. In the default position the articulated mouth mask 900 covers the mouth of one embodiment.

### An Articulated Mouth Mask Raised:

FIG. 10B shows for illustrative purposes only an example of an articulated mouth mask raised of one embodiment. FIG. 10B shows the user 110 with the user's mouth opened. The nasal mask 100 is seen being worn by the user 110 with the articulated mouth mask raised 1000 coupled to the nasal mask 100. In this example the articulated mouth mask raised 1000 is in an up position when the user opens their mouth. A chin strap 1010 is coupled to the nasal mask 100 and extends up to couple to the top of the articulated mouth mask 900 of FIG. 9. When the chin is lowered to open the mouth the chin strap 1010 pulls on the top of the articulated mouth mask 900 of FIG. 9 to raise it up from the default position. Upon closing the mouth the chin strap 1010 is relaxed and the articulated mouth mask 900 of FIG. 9 is lowered to the default position of one embodiment.

### Nasal Spray Application:

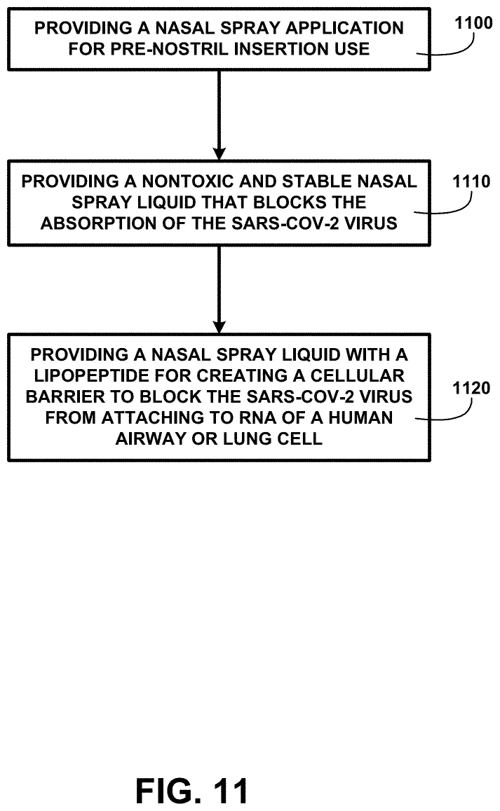

FIG. 11 shows a block diagram of an overview of a nasal spray application of one embodiment. FIG. 11 shows a nasal spray application to block SARS-COV-2 virus from attaching to human cells. Providing a nasal spray application for pre-nostril insert insertion use 1100. Providing A nontoxic and stable nasal spray liquid that blocks the absorption of the SARS-CoV-2 virus 1110 preventing a user from being infected. Providing a nasal spray liquid with a lipopeptide for creating a cellular barrier to block the SARS-CoV-2 virus from attaching to RNA of a human airway or lung cell 1120. This disables the SARS-CoV-2 virus from infecting the user and suffering the symptoms associated with COVID-19 of one embodiment.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1. A method, comprising:
fabricating a nasal mask configured for preventing nasal inhaling of airborne Covid-19 virus;
making the nasal mask using a breathable fabric configured for creating a breathable fabric filter for filtering out air borne viruses;
providing at least two ear straps coupled to the nasal mask configured for a user looping the at least two ear straps over the user's ears;
coating the breathable fabric filter with a disinfectant for inactivating the Covid-19 virus:
integrating a dorsum conforming nose piece coupled to the nasal mask configured for creating a gap free fit; and
fabricating structural stitching in the nasal mask configured for maintaining a breathable space fronting the nostrils.

2. The method of claim 1, further comprising using multiple structural stitching in the nasal mask configured for creating an upper lip section configured for providing an upper lip no gap fit for preventing unfiltered air from being inhaled through the nostrils.

3. The method of claim 1, further comprising providing a pliable metal strip coupled to the dorsum conforming nose piece configured for a user pressing the pliable metal strip against the nose dorsum and sides for forming a gap free fit.

4. The method of claim 1, further comprising creating multiple structural stitching in the nasal mask configured for reinforcing the shape of the nasal mask area in front of the nostrils.

5. The method of claim 1, further comprising using multiple structural stitching in the nasal mask configured for creating face fitting no gap side panels for preventing unfiltered air from being inhaled through the nostrils.

6. (canceled)

7. The method of claim 1, further comprising coating the breathable fabric filter with an antiseptic povidone-iodine solution ranging from 0.5% to 2.5% concentration for inactivating the Covid-19 virus within 15 seconds.

8. The method of claim 1, further comprising coupling two nostril inserts with a mask space extending structure to the nasal mask configured for providing an open weave anti-microbial barrier in each nostril.

9. The method of claim 1, further comprising coupling a short-range wireless communications technology including a Bluetooth® device to each of the at least two ear straps for receiving an audible alert that a coronavirus has been detected.

10. The method of claim 1, further comprising coupling a virus detection sensor with near-field transmitter to the nasal mask for detecting the presence of a coronavirus and transmitting an audible alert that a coronavirus has been detected to a short-range wireless communications technology including a Bluetooth® device.

11. An apparatus, comprising:
a nasal mask configured for preventing nasal inhaling of airborne Covid-19 virus;
a breathable fabric provided in the nasal mask configured for creating a breathable fabric filter for filtering out air borne viruses;
a coating applied to the breathable fabric filter with a disinfectant for inactivating the Covid-19 virus;
at least two ear straps coupled to the nasal mask configured for a user looping the at least two ear straps over the user's ears;
a dorsum conforming nose piece coupled to the nasal mask configured for creating a gap free fit; and
structural stitching coupled in the nasal mask configured for maintaining a breathable space fronting nostril nasal passages.

12. The apparatus of claim 11, further comprising multiple structural stitching in the nasal mask configured for creating an upper lip section configured for providing an upper lip no gap fit for preventing unfiltered air from being inhaled through the nostrils.

13. The apparatus of claim 11, further comprising a pliable metal strip coupled to the dorsum conforming nose piece configured for a user pressing the pliable metal strip against the nose dorsum and sides for forming a gap free fit.

14. (canceled)

15. The apparatus of claim 11, further comprising multiple structural stitching in the nasal mask configured for creating face fitting no gap side panels for preventing unfiltered air from being inhaled through the nostrils.

16. An apparatus, comprising:
a breathable fabric filter configured for creating a nasal mask for preventing nasal inhaling of airborne SARS-CoV-2 virus;
at least two ear straps configured for a user looping the at least two ear straps over the user's ears for wearing the nasal mask;
a coating applied to the breathable fabric filter with a disinfectant inactivating the SARS-CoV-2 virus;
a dorsum conforming nose piece creating a gap free fit coupled to the nasal mask; and
structural stitching configured for maintaining a breathable space fronting nostril nasal passages coupled to the nasal mask.

17. The apparatus of claim 16, further comprising an upper lip section coupled to the nasal mask configured for providing an upper lip no gap fit for preventing unfiltered air from being inhaled through the nostrils.

18. The apparatus of claim 16, further comprising a left and right face fitting side panel coupled to the nasal mask configured to prevent unfiltered air from being inhaled through the nostrils.

19. The apparatus of claim 16, further comprising coupling electronic devices to the nasal mask configured to detect the presence of a coronavirus and transmitting an audible signal to a receiver for broadcasting an audible alert to a user.

20. The apparatus of claim 16, further comprising at least two nostril inserts with an anti-microbial coating configured to form an open weave anti-microbial barrier for inactivating air borne SARS-CoV-2 virus before entering the nasal cavity.
